(19) United States
(12) Patent Application Publication
(10) Pub. No.: US 2009/0131751 A1
(43) Pub. Date: May 21, 2009

(54) Anal surgical instrument guides

(76) Inventors: James T. Spivey, Cincinnati, OH (US); William B. Weisenburgh, II, Maineville, OH (US)

Correspondence Address: K&L GATES LLP, 535 SMITHFIELD STREET, PITTSBURGH, PA, 15222, US

(21) Appl. No.: 11/986,084

(22) Filed: Nov. 20, 2007

(51) Int. Cl. A61B 1/01

(52) U.S. Cl. 600/114

(57) ABSTRACT

A surgical instrument guide configured to be at least partially inserted into a natural orifice of a patient to allow a surgeon to access a tissue treatment region with multiple surgical instruments during the course of a minimally invasive surgical procedure. In various embodiments, the surgical instruments can be inserted at various angles with respect to the tissue treatment region to provide the surgeon with better access to the surgical site during an operation. In at least one embodiment, the surgical instrument guide may include a body portion having a first port and a second port defined therein. In such embodiments, the first and second ports may each be configured to receive a surgical instrument therethrough. Additionally, in various embodiments, at least one of the first and second ports can be configured to allow for pivotal movement of the surgical instrument within the body of the surgical instrument guide.

### BACKGROUND

i. Field of the Invention

The present invention generally relates to surgical instrument guides, and to surgical instrument guides which can be at least partially inserted into a patient during a surgical procedure.

ii. Description of the Related Art

Traditional, or open, surgical techniques may require a surgeon to make large incisions in a patient's body in order to access a tissue treatment region, or surgical site. In some instances, these large incisions may prolong the recovery time of and/or increase the scarring to the patient. As a result, minimally invasive surgical techniques are becoming more preferred among surgeons and patients owing to the reduced size of the incisions required for various procedures. In some circumstances, minimally invasive surgical techniques may reduce the possibility that the patient will suffer undesirable post-surgical conditions, such as scarring and/or infections, for example. Further, such minimally invasive techniques can allow the patient to recover more rapidly as compared to traditional surgical procedures.

Endoscopy is one minimally invasive surgical technique which allows a surgeon to view and evaluate a surgical site by inserting at least one cannula, or trocar, into the patient's body through a natural body opening and/or through a relatively small incision. In use, an endoscope can be inserted into, or through, the trocar so that the surgeon can observe the surgical site. In various embodiments, the endoscope may include a flexible or rigid shaft, a camera and/or other suitable optical device, and a handle portion. In at least one embodiment, the optical device can be located on a first, or distal, end of the shaft and the handle portion can be located on a second, or proximal, end of the shaft. In various embodiments, the endoscope may also be configured to assist a surgeon in taking biopsies, retrieving foreign objects, and introducing surgical instruments into the surgical site.

Laparoscopic surgery is another minimally invasive surgical technique where procedures in the abdominal or pelvic cavities can be performed through small incisions in the patient's body. A key element of laparoscopic surgery is the use of a laparoscope which typically includes a telescopic lens system that can be connected to a video camera. In various embodiments, a laparoscope can further include a fiber optic system connected to a halogen or xenon light source, for example, in order to illuminate the surgical site. In various laparoscopic, and/or endoscopic, surgical procedures, a body cavity of a patient, such as the abdominal cavity, for example, can be insufflated with carbon dioxide gas, for example, in order to create a temporary working space for the surgeon. In such procedures, a cavity wall can be elevated above the organs within the cavity by the carbon dioxide gas. Carbon dioxide gas is usually used for insufflation because it is easily absorbed and removed by the body.

In at least one minimally invasive surgical procedure, an endoscope and/or laparoscope can be inserted through a natural opening of a patient to allow a surgeon to access a surgical site. Such procedures are generally referred to as Nature Orifice Transluminal Endoscopic Surgery or (NOTES)™ and can be utilized to treat tissue while reducing the number of incisions, and external scars, to a patient's body. In various NOTES procedures, for example, an endoscope can include at least one working channel defined therein which can be used to allow the surgeon to insert a surgical instrument therethrough.

### SUMMARY

In at least one form of the invention, a surgical instrument guide can be at least partially positioned within a natural orifice of a patient in order to aid a surgeon in accessing a tissue treatment region, or surgical site, during the course of a minimally invasive surgical procedure. In various embodiments, a plurality of surgical instruments can be inserted into the guide at various angles relative to each other and/or relative to the surgical site. In at least one embodiment, the surgical instrument guide can include a body portion having at least a first port and a second port defined therein wherein each of the first and second ports can be configured to receive at least one surgical instrument therein. In various embodiments, at least one of the first and second ports can include a first end in which a surgical instrument can be inserted therein and, in addition, a second end in which at least a portion of the surgical instrument can exit the guide and access a tissue treatment region. In at least one embodiment, at least one of the first and second ports can include a substantially conical profile which can be configured to allow a surgical instrument to be pivoted within the port. In addition, in various embodiments, at least one of the first and second ports can be configured to pivot, or otherwise suitably move, relative to the other port.

In at least one form of the invention, a surgical instrument guide can include a body having a first portion and a second portion, wherein the first portion can be configured to be positioned outside of an anus of a patient, and wherein the second portion can be configured to be positioned within the patient's colonic cavity. In various embodiments, the guide can further include a neck portion positioned intermediate the first and second portions wherein the neck portion can be configured to be received within the anus of the patient such that the anal sphincter can contract around the neck portion. In at least one such embodiment, as a result, the second portion can be held within the colonic cavity while the surgeon utilizes ports within the guide to access the surgical site.

In at least one form of the invention, a surgical instrument guide can include an elongate member which can be moved between a first, or undeployed, position and a second, or deployed, position. In various embodiments, the elongate member can include a first portion and a second portion, wherein the first and second portions can each be configured to receive at least one surgical instrument therein. In at least one embodiment, the first portion can define a first axis and the second portion can define a second axis. In at least one such embodiment, the first axis and the second axis can be oriented in a first relative position when the elongate member is in an undeployed configuration and, in addition, the first axis and the second axis can be oriented in a second relative relationship when the elongate member is in a deployed configuration.

### BRIEF DESCRIPTION OF THE DRAWINGS

The above-mentioned and other features and advantages of the various embodiments of this invention, and the manner of attaining them, will become more apparent and the invention itself will be better understood by reference to the following description of embodiments of the invention taken in conjunction with the accompanying drawings, wherein:

FIG. 1 is a view of a surgical instrument guide partially positioned within a natural orifice of a patient in accordance with one non-limiting embodiment of the present invention;

FIG. 2 is a perspective view of the surgical instrument guide of FIG. 1;

FIG. 3 is another perspective view of the surgical instrument guide of FIG. 1;

FIG. 4 is a front view of the surgical instrument guide of FIG. 1;

FIG. 5 is a rear view of the surgical instrument guide of FIG. 1;

FIG. 6 is a side view of a surgical instrument guide having a tissue-piercing portion in accordance with one non-limiting embodiment of the present invention;

FIG. 7 is a perspective view of a surgical instrument guide having a tissue-piercing portion in accordance with one alternative non-limiting embodiment of the present invention;

FIG. 8 is a rear view of a surgical instrument guide in accordance with another non-limiting embodiment of the present invention;

FIG. 9 is a cross-sectional view of the surgical instrument guide of FIG. 8;

FIG. 10 is another cross-sectional view of the surgical instrument guide of FIG. 8 having a flapper-type seal;

FIG. 11 is a front view of the surgical instrument guide of FIG. 8 having a flapper-type seal;

FIG. 12 is rear view of a surgical instrument guide in accordance with another non-limiting embodiment of the present invention;

FIG. 13 is a cross-sectional view of the surgical instrument guide of FIG. 12 illustrating an instrument shaft within a port;

FIG. 14 is another cross-sectional view of the surgical instrument guide of FIG. 12 illustrating the instrument in a different orientation;

FIG. 15 is a front view of the surgical instrument guide of FIG. 12;

FIG. 16 is a view of a surgical instrument guide in an undeployed position where the guide is situated at least partially through a patient in accordance with one non-limiting embodiment of the present invention;

FIG. 17 is a view of the surgical instrument guide of FIG. 16 in a deployed position;

FIG. 18 is a perspective view of the surgical instrument guide of FIG. 16 in a deployed position;

FIG. 19 is a perspective view of a first instrument receiving portion of the surgical instrument guide of FIG. 16; and

FIG. 20 is a perspective view of a second instrument receiving portion of the surgical instrument guide of FIG. 16.

Corresponding reference characters indicate corresponding parts throughout the several views. The exemplifications set out herein illustrate preferred embodiments of the invention, in one form, and such exemplifications are not to be construed as limiting the scope of the invention in any manner.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the various embodiments of the present invention is defined solely by the claims. The features illustrated or described in connection with one exemplary embodiment may be combined with the features of other embodiments. Such modifications and variations are intended to be included within the scope of the present invention.

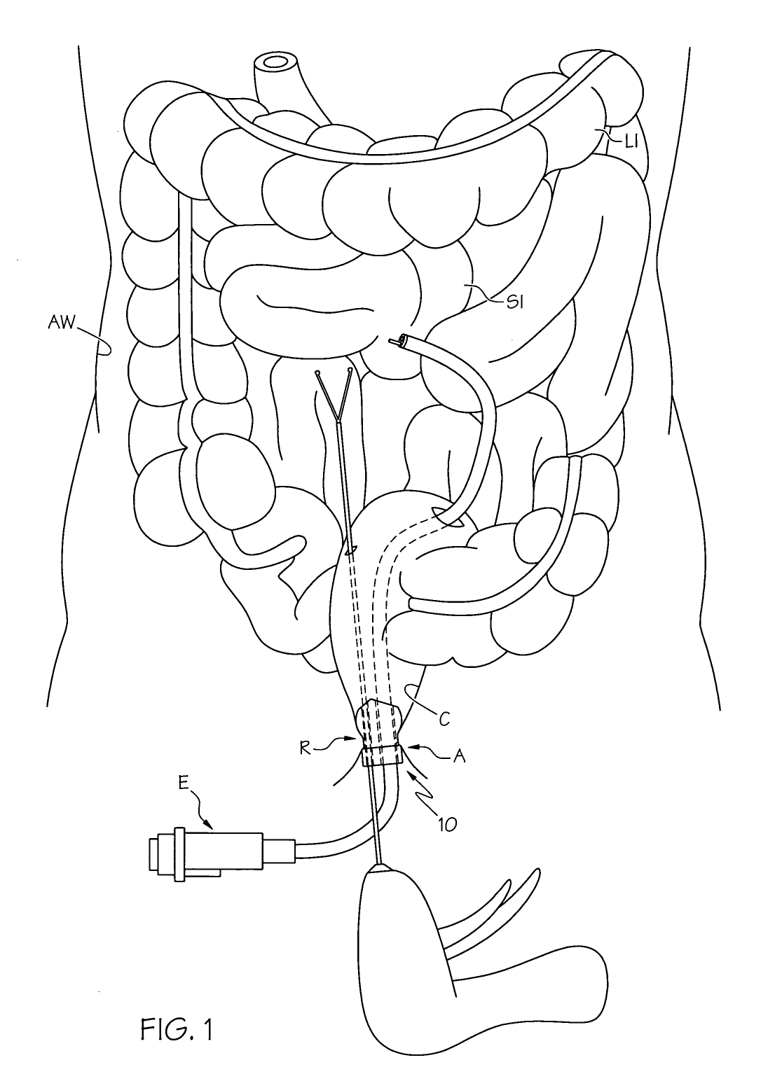

During the course of various surgical procedures, there often exists a need to manipulate, grasp, move, lift, pull, twist, and/or push various organs within a patient's body, especially in intraluminal and transluminal procedures. Performing these functions on heavy and/or affixed organs at the distal end, or end-effector, of long and/or flexible surgical instruments can pose an especially challenging set of difficulties. Among the greatest difficulties is addressing the need to introduce a surgical instrument into a tissue treatment region via a working channel in an endoscope “E” (FIG. 1) wherein the working channel is typically no larger than a few millimeters in diameter. Further complicating this challenge is the fact that the organs to be moved and manipulated, such as the liver and the spleen, for example, are often floppy, large and relatively dense which makes them difficult to control. Other organs are somewhat friable and heavily vascular and, as a result, must be handled very carefully. Still other organs, such as the large intestine “LI” and small intestine “SI”, for example, can be difficult to work with because of their size and flexibility. These challenges create the need for a surgical instrument guide which can be configured to permit a surgeon to insert multiple surgical instruments, including an endoscope, into the same natural orifice of a patient's body in order to increase the surgeon's access to a surgical site.

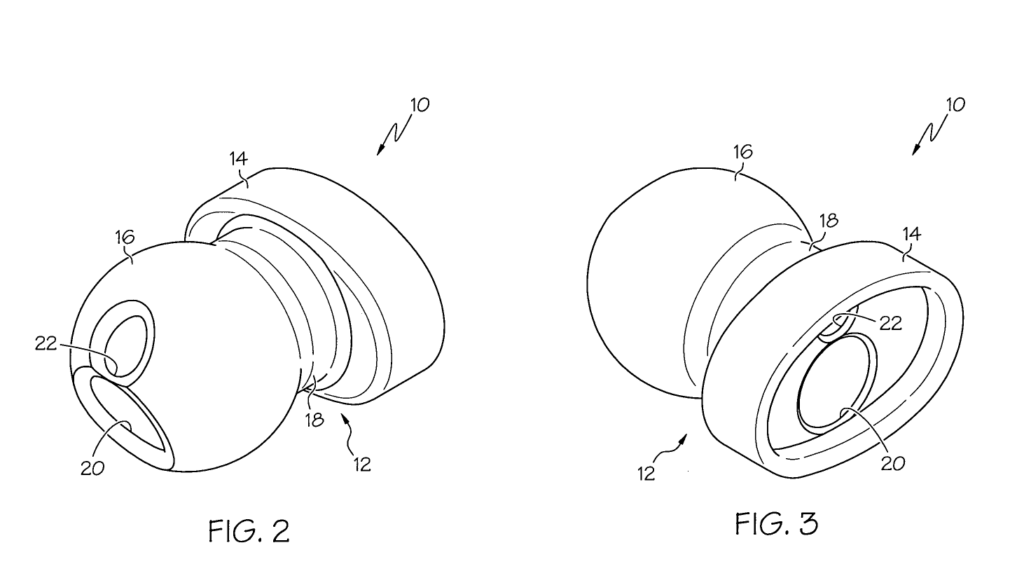

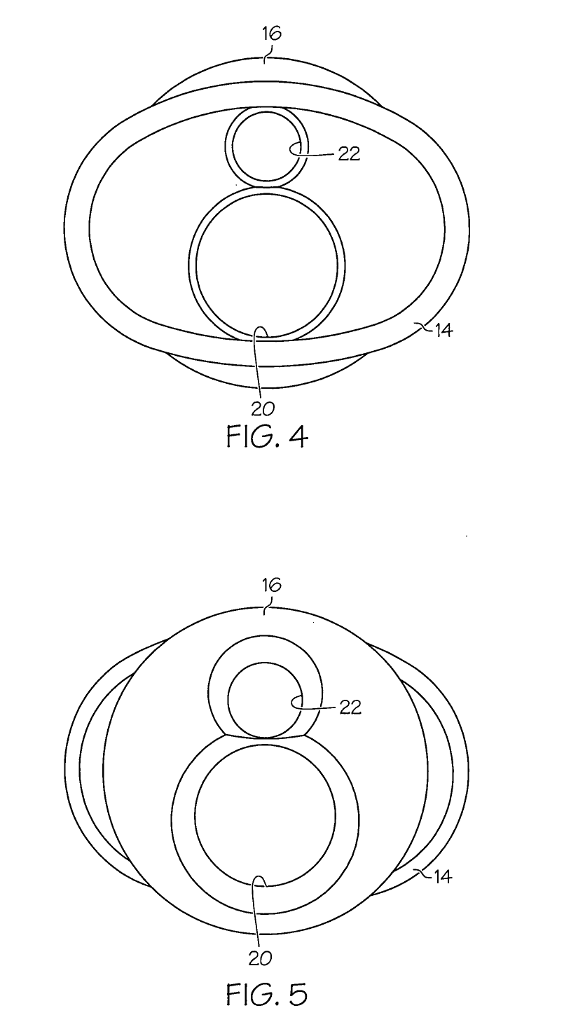

In various embodiments, a surgical instrument guide can be utilized to insert multiple surgical instruments into a colonic cavity “C” of a patient through the patient's anus “A”. In at least one embodiment, referring to FIGS. 1-5, surgical instrument guide 10 can include body 12 having first portion 14, second portion 16, and neck portion 18. In various embodiments, second portion 16 can be configured to be inserted into the colonic cavity of a patient through their anus and, in at least one embodiment, first portion 14 can be configured to be positioned external to the colonic cavity. In at least one embodiment, second portion 16 can be substantially spherical in shape such that it can be slid through the opening in the anus and into the colonic cavity without injuring the patient. In various alternative embodiments, second portion 16 can include any other suitable shape.

Further to the above, in various embodiments, first portion 14 can be configured such that the proper position of guide 10 within the colonic cavity can be reliably achieved. More particularly, in at least one embodiment, first portion 14 can be configured to abut the external portion of the anus of the patient to stop guide 10 from being inserted further into the colonic cavity. In various embodiments, first portion 14 can be substantially oval and/or elliptical shaped, for example, wherein first portion 14 can be configured to be grasped by the surgeon. In at least one such embodiment, as described in greater detail below, the surgeon can rotate guide 10 within the colonic cavity by rotating first portion 14. In various other embodiments, first portion 14 can have any suitable shape configured to assist the surgeon in positioning and manipulating guide 10 within the colonic cavity. In various embodiments, surgical instrument guide 10 can be comprised of any suitable rigid, semi-rigid, and/or flexible material, such as polycarbonate, PEEK, PTFE, silicone, urethane, and/or polyolefin, for example.

In various embodiments, neck portion 18 of body 12 can be situated intermediate first portion 14 and second portion 16 and can have a smaller cross-section, or diameter, than the first and second portions. Stated another way, first portion 14 can define a first perimeter, second portion 16 can define a second perimeter, and neck portion 18 can define a perimeter which is smaller than the first and second perimeters. In at least one embodiment, when second portion 16 is suitably positioned within the colonic cavity, neck portion 18 can be positioned within the anus of the patient. In various embodiments, the patient's anal sphincter can contract around neck portion 18 to hold guide 10 in position during the surgical procedure. In at least one embodiment, neck portion 18 can include a groove configured to receive at least a portion of the anal sphincter. In various circumstances, owing to the position of the anal sphincter around neck 18, and also owing to the larger perimeters, or diameters, of first portion 14 and second portion 16, guide 10 can be retained within the colonic cavity and the possibility of guide 10 being unintentionally removed from the colonic cavity can be reduced. In various embodiments, neck portion 18 can be generally circular, elliptical, and/or oval in shape such that it can be rotated within the anus of the patient without causing injury to the patient.

In various embodiments, surgical instrument guide 10 can further include at least a first port and a second port defined therethrough, wherein each port can be configured to receive at least a portion of a surgical instrument therein. In at least one embodiment, referring to FIGS. 2-5, first port 20 and second port 22 can be defined through first portion 14, second portion 16, and neck portion 18 of body 12. In various embodiments, each of first port 20 and second port 22 can include an aperture extending through body 12, wherein each aperture can include a first end configured to receive a surgical instrument and, in addition, a second end configured to allow at least a portion of the surgical instrument to extend therethrough. In at least one embodiment, the apertures can be configured to slidably receive the surgical instruments and provide a pathway for the surgeon to access a surgical site. In various embodiments, at least one of ports 20 and 22 can be configured such that there is clearance between the sidewalls of the aperture and the surgical instrument positioned therein. In other embodiments, at least one of ports 20 and 22 can be configured such that the surgical instrument can engage the sidewalls of the aperture. In at least one such embodiment, the port can be configured to align the surgical instrument along a pre-determined axis, for example.

In use, a surgeon can insert second portion 16 of body 12 into the anus of the patient until the anal sphincter surrounds neck portion 18 and the sphincter contracts around neck portion 18 as described above. In at least one embodiment, a lubricant can be applied to the outer surfaces of second portion 16 and/or neck portion 18, for example, in order to reduce the coefficient of friction between guide 10 and the anus and/or rectum “R” of the patient, for example, and facilitate the insertion of guide 10 into the colonic cavity. In either event, in at least one embodiment, the surgeon can then insert an endoscope through first port 20, for example, and a surgical instrument through second port 22 such that the surgeon can view the tissue treatment region, or surgical site, with the endoscope while working within the surgical site with the surgical instrument. In at least one such embodiment, the endoscope can include a working channel therein configured to at least partially receive a second surgical instrument such that at least a portion of the second surgical instrument can be positioned within the surgical site.

In various embodiments, such surgical instruments can include one or more of an endocutter, an endoscope, a light, a viewing instrument or camera, scissors, graspers, forceps, a cutting instrument, a harmonic instrument, an RF instrument, and/or an insufflation pressure control instrument, for example. Of course, depending on the particular surgical procedure being performed, other suitable surgical instruments can also be used. In various embodiments, a plurality of surgical instruments can be inserted into any one of the first and second ports in the body of the surgical instrument guide. In at least one embodiment, a surgical instrument guide can have more than two ports and the surgeon can insert additional instruments into the surgical site through these additional ports in the same or a similar manner as described above.

In various embodiments, a surgical instrument guide in accordance with at least one embodiment of the present invention can be configured to prevent, or at least reduce the possibility of, the anus and/or rectum of a patient, for example, being torn or otherwise damaged by a surgical instrument. More particularly, absent a surgical instrument guide described herein, a surgical instrument inserted into a colonic cavity through the anus of a patient may directly contact the anus and cause damage thereto, especially when the surgical instruments are removed from and re-inserted into the surgical site several times during a procedure. In various circumstances, the insertion and sliding of a surgical instrument within the anus can create friction therebetween and can complicate the use of the surgical instruments. In at least one embodiment, the sidewalls of ports 20 and 22, for example, can eliminate, or at least reduce, contact between the surgical instruments and the anus and/or rectum, for example. Furthermore, the sidewalls of ports 20 and 22 can be configured to provide an appropriate amount of friction between the surgical instruments and the surgical instrument guide. More particularly, in at least one embodiment, the sidewalls of ports 20 and 22 can include a surface texture, coating, and/or surface roughness which can allow the surgical instruments to be easily slid therethrough yet provide a sufficient amount of static friction to prevent, or at least inhibit, the surgical instruments from moving unintentionally. Owing to these advantages, first and second ports 20 and 22 of guide 10 can facilitate the insertion, removal, and re-insertion of a surgical instrument, or different surgical instruments, into a surgical site and also, as outlined above, reduce the possibility of injury to the patient. In various embodiments, first and second ports 20 and 22 can be lubricated to reduce the coefficient of friction between the sidewalls of the ports and the surgical instruments.

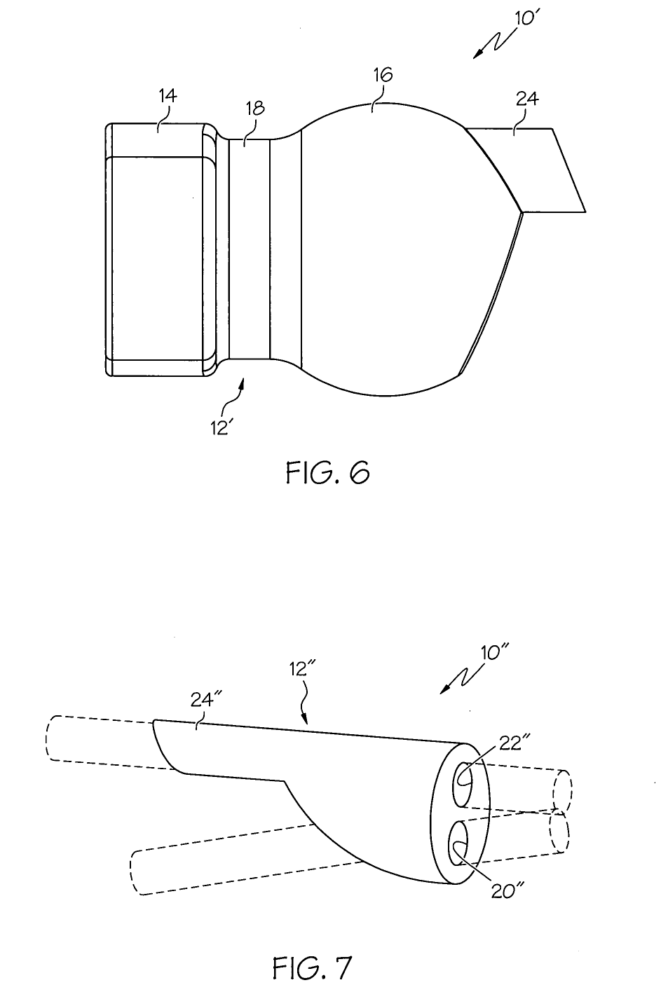

In various embodiments, referring to FIG. 6, body 12′ of guide 10′ can include tissue-piercing portion 24 which can be configured to pierce the sidewall of a body cavity and/or organ, for example. In various circumstances, such embodiments may be useful during a transluminal procedure, for example, to allow a surgeon to pierce tissue and access a tissue treatment region. In at least one embodiment, tissue-piercing portion 24 can have features, such as ribs, for example, on an outer surface. In such an embodiment, the features can be configured to retain tissue-piercing portion 24 in frictional-engagement with the pierced tissue to prevent tissue-piercing portion 24 from sliding during the insertion and/or retraction of a surgical instrument. In various embodiments, tissue-piercing portion 24 can be attached to, in sliding contact with, mounted to, and/or integrally-formed with second portion 16, for example, and extend outwardly therefrom. In at least one embodiment, tissue-piercing portion 24 can be axially aligned with or otherwise situated over at least one of first and second ports 20 and 22 such that at least one of the surgical instruments can be slid through the port and the tissue-piercing portion to access the tissue treatment region. In various embodiments, tissue-piercing portion 24 can be at least partially inserted into the anus and the colonic cavity before second portion 16 is attached thereto.

In other various embodiments, referring to FIG. 7, guide 10″ can include body 12″ and tissue-piercing portion 24″. In at least one embodiment, body 12″ can be configured such that it can be at least partially inserted into a natural cavity of a patient. In various embodiments, tissue-piercing portion 24″ can be useful during a transluminal procedure, for example, to allow a surgeon to pierce tissue and access a tissue treatment region. Much like tissue-piercing portion 24 described above, tissue-piercing portion 24″ can be axially aligned with at least one of first port 20″ and second port 22″ such that a surgical instrument can be slid through one of the ports and tissue-piercing portion 24″ to access the tissue treatment region. In various embodiments, a first shaft of a first surgical instrument situated within first port 20″ can be transverse or skew to a second shaft of a second surgical instrument situated within second port 22″, for example.

In various embodiments, referring to FIGS. 2-5, first port 20 can define a first axis along which a first surgical instrument can be oriented and second port 22 can define a second axis along which a second surgical instrument can be oriented. In at least one embodiment, the first axis can be parallel to the second axis. In other various embodiments, the first axis can be transverse, skew, or perpendicular to the second axis such that, when surgical instruments are positioned within the first and second ports, the surgical instruments can be oriented in different directions. Such embodiments can be useful in procedures in which tissue must be manipulated, for example, in more than one tissue treatment region. Such embodiments can also be useful in procedures during which it may be necessary to triangulate two or more surgical instruments within a surgical site. In various embodiments, surgical instrument guide 10, for example, can be configured to be rotated within a patient's colonic cavity in order to re-orient or re-align the axes of the first and second ports such that the surgical instruments situated within the first and second ports can be better positioned within a surgical site.

In various embodiments, as outlined above, a surgeon can move a surgical instrument within a port in a surgical instrument guide to better access a surgical site. In at least one embodiment, referring to FIGS. 8-11, at least one of first port 120 and second port 122, for example, can be configured such that a surgical instrument can be pivoted, tilted, or rotated, within the port. In various embodiments, first port 120 can include an aperture having a first portion and a second portion, wherein the first portion can define a perimeter which can be larger than a perimeter of the second portion. In such embodiments, the larger perimeter of the first portion can allow a surgical instrument to be pivoted, or otherwise angulated, within first port 120. In at least one embodiment, at least a portion of first port 120 can include substantially conically-shaped chamfer, or portion, 126, wherein conically-shaped portion 126 can include the first and second perimeters described above. In various embodiments, referring to FIG. 10, first port 120′ can include a third portion having a third perimeter, wherein the third perimeter can be larger than the second perimeter. In at least one such embodiment, first port 120′ can include a second substantially conically-shaped portion 127 including the second perimeter and the third perimeter described above. In various embodiments, such an additional conically-shaped portion can increase the range in which a surgical instrument can be pivoted within port 120′.

In various circumstances, further to the above, a surgical instrument can be pivoted within a port until at least a portion of the surgical instrument abuts a side wall of the port. In such circumstances, a surgeon may be prohibited from further pivoting, or angulating, the surgical instrument within the port. In at least one embodiment, however, the port, or at least portions thereof, can be configured to move relative to a body of the surgical instrument guide such that the surgical instrument can be pivoted, or angulated, even though the surgical instrument is abutting, or positioned adjacent to, the side wall of the port. In various embodiments, such a movable port can provide a surgeon with the ability to adjust the surgical instrument angularly without moving the guide. In at least one embodiment, a movable first port can allow a surgeon to move a first surgical instrument relative to a second surgical instrument within a second port, for example. In various embodiments, a surgical instrument guide can include more than one movable port.

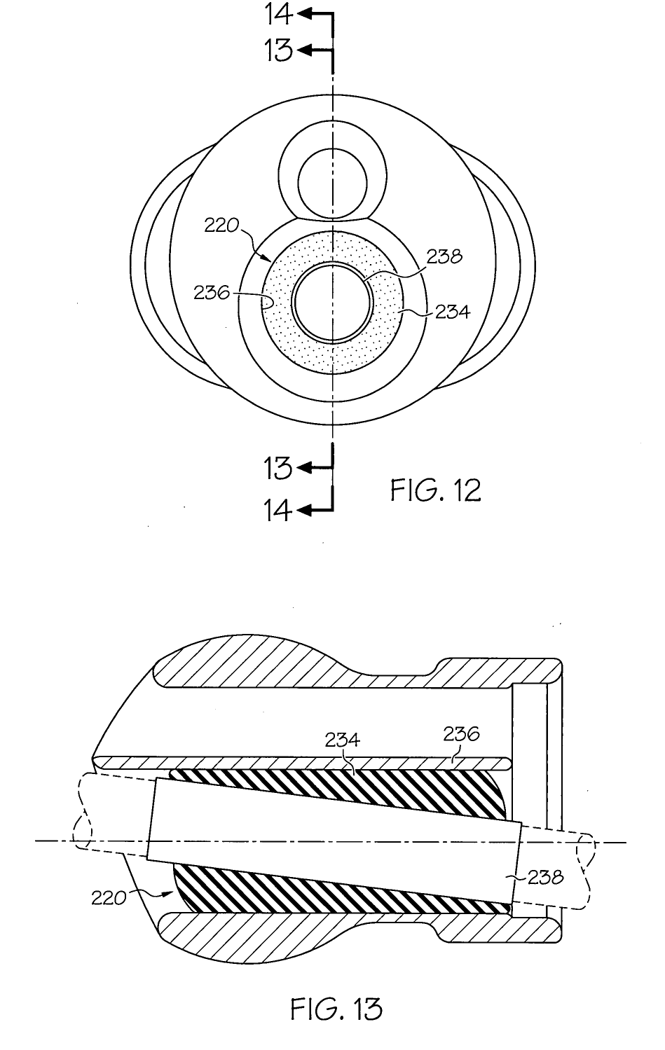

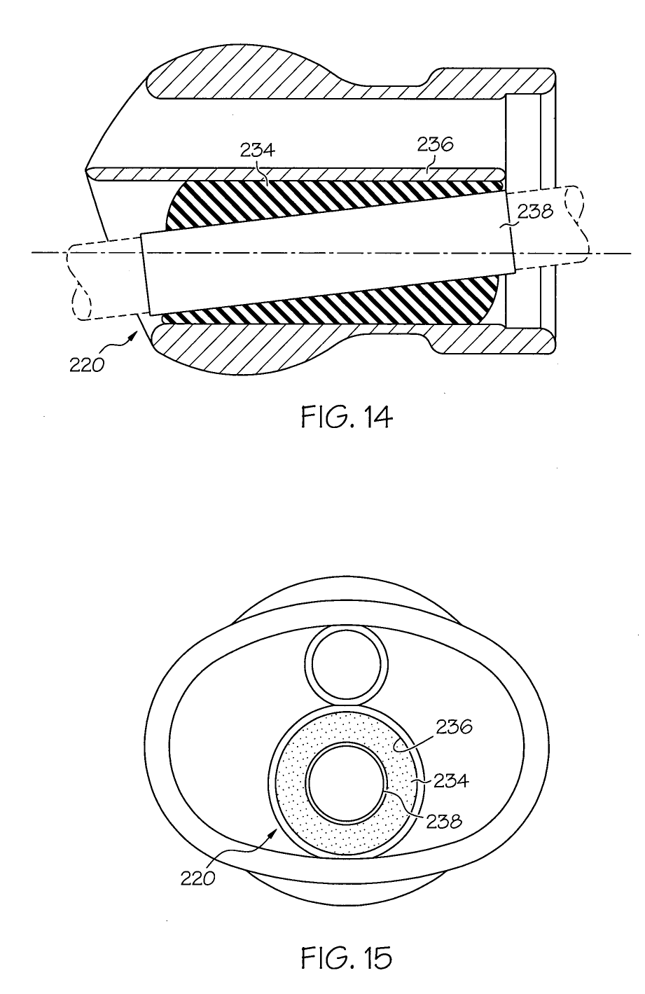

In various embodiments, referring to FIGS. 12-15, first port 220 can comprise outer shell 236, inner shell 238, and resilient material 234. In at least one embodiment, inner shell 238 can be configured to move within outer shell 236 wherein resilient material 234 can be configured to expand and/or contract to accommodate such relative movement. In such an embodiment, resilient material 234 can also act as a seal between an outside environment and the tissue treatment region, for example. In various embodiments, resilient material 234 can be positioned intermediate outer shell 236 and inner shell 238 such that outer shell 236 and inner shell 238 can both contact resilient material 234. In various embodiments, resilient material 234 can be comprised of foam, rubber, gel, and/or a gel-like substance, for example. In use, a surgeon can articulate a surgical instrument positioned within first port 220 by applying a force to an end of the surgical instrument and rotating inner shell 238 relative to outer shell 236. In various embodiments, a surgical instrument guide can include a ball and socket joint, for example. In at least one embodiment, the ball and socket joint can include a ball portion which can at least partially fit within a cavity of a socket portion such that the ball portion can rotate within the socket portion. In at least one such embodiment, the ball portion can be configured to rotate with respect to the socket portion about a plurality of axes. In various embodiments, the ball portion can include an aperture extending at least partially therethrough wherein the aperture can be configured to allow an instrument to be disposed therein.

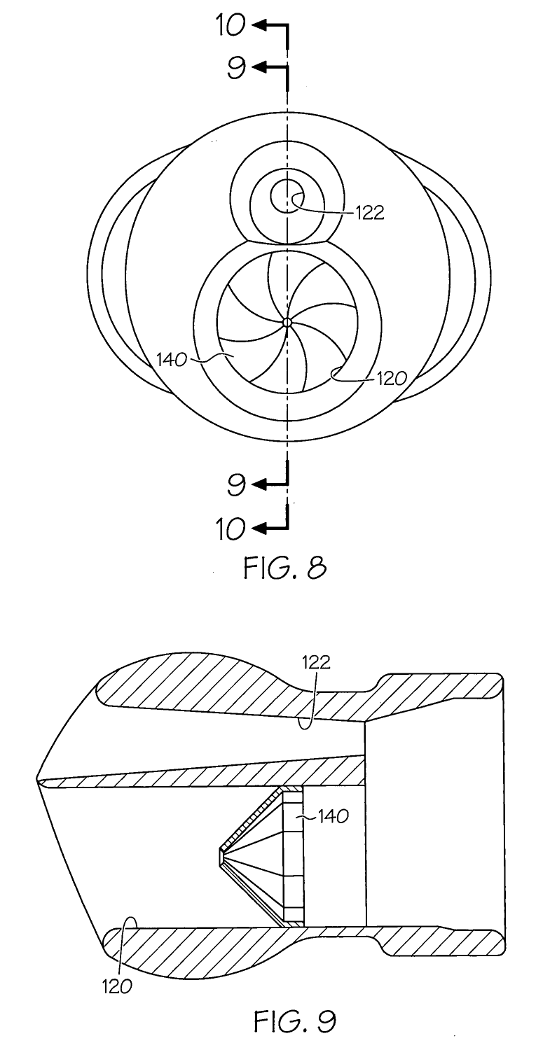

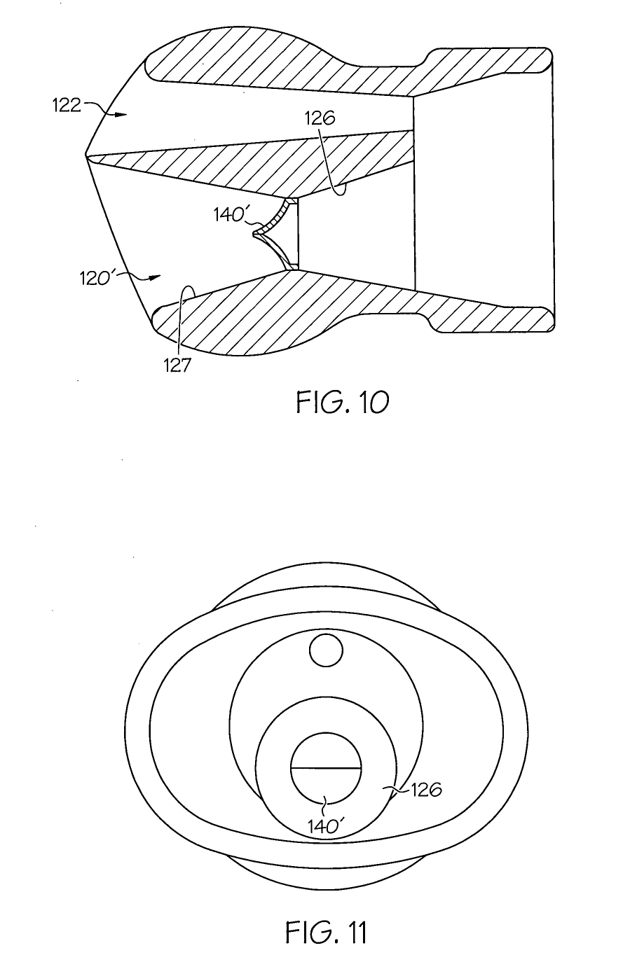

In various embodiments, at least one seal can be disposed within at least one of the ports such that an at least substantially sealed interface can be created between the surgical instrument guide and a surgical instrument positioned therein. In various embodiments, referring to FIGS. 8 and 9, seal 140 can be configured to create a seal between the surgical instrument and first port 120, for example, to prevent, or at least inhibit, a fluid from entering into or escaping from the patient's body. In at least one embodiment, seal 140 can be configured to maintain the insufflation within a patient during a surgical procedure. In various embodiments, a surgical instrument guide can include a seal positioned within the entrance to port 120 and/or a seal positioned within the exit to port 120. In at least one embodiment, a seal can be positioned intermediate the first and second conically-shaped portions of port 120, for example. In various embodiments, a seal can be included on the shaft of a surgical instrument. In other various embodiments, a first seal can be included on the shaft of a surgical instrument and a second seal can be included within a port. In various embodiments, seal 140 can be comprised of any suitable material, such as poly-isoprene and/or silicone, for example. In various embodiments, referring to FIGS. 10 and 11, seal 140′ can be configured as a flapper-type seal and can be attached to, and/or integrally formed with, a portion of the inner diameter of the port, for example. Examples of seals which can be used with the present invention are illustrated in U.S. Pat. No. 5,401,248, entitled SEAL FOR TROCAR ASSEMBLY, issued on Mar. 28, 1995, U.S. Pat. No. 5,628,732, entitled TROCAR WITH IMPROVED UNIVERSAL SEAL, issued on May 13, 1997, and U.S. Pat. No. 5,792,113, entitled UNIVERSAL SEAL FOR A TROCAR, issued on Aug. 11, 1998, the entire disclosures of which are hereby incorporated by reference.

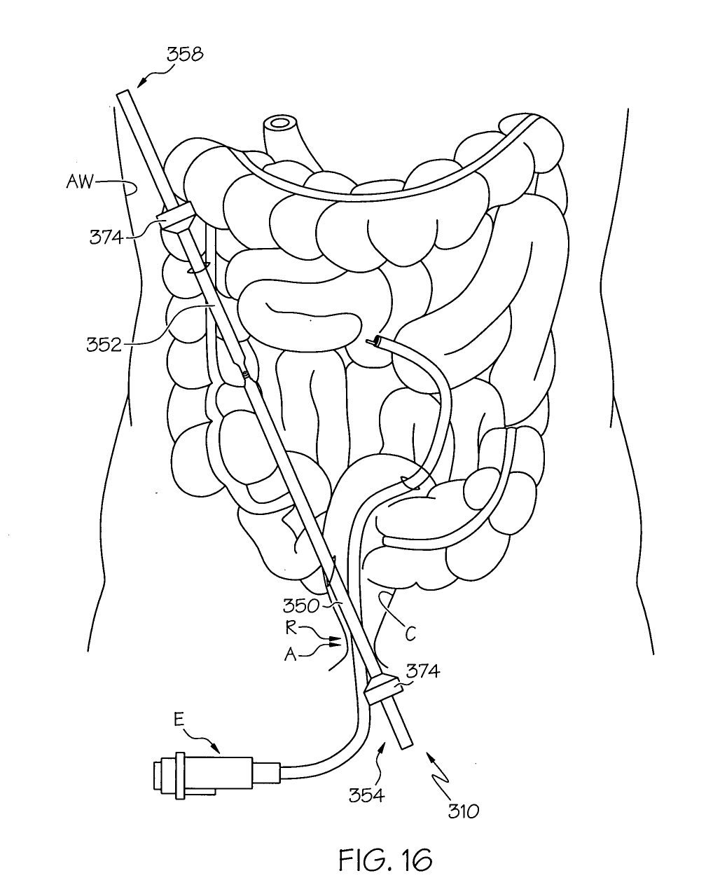

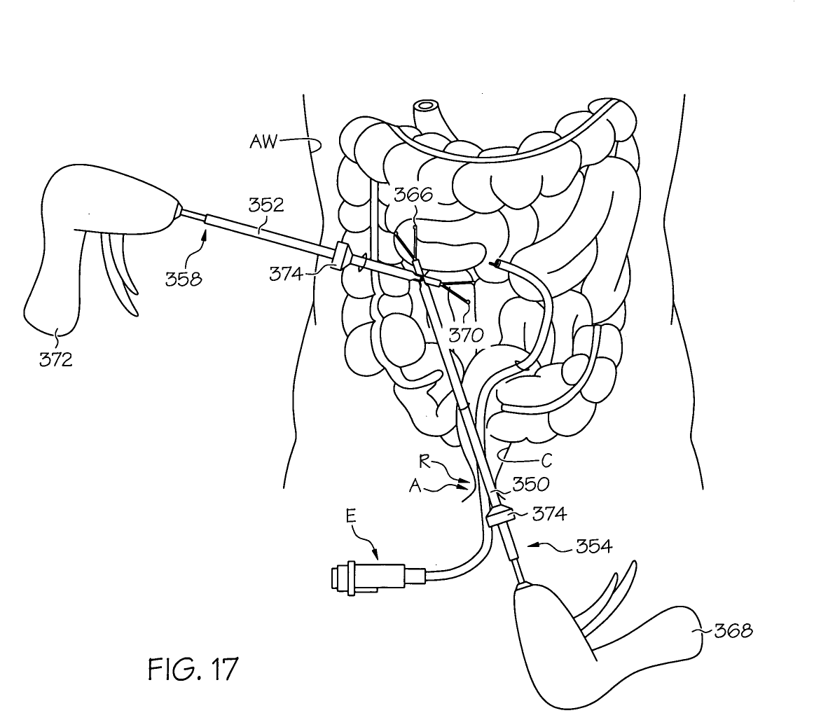

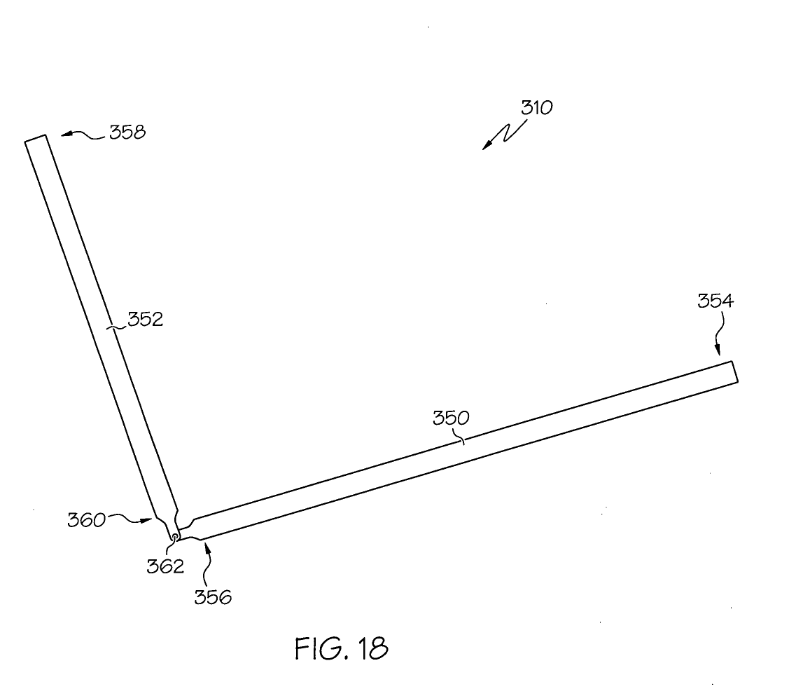

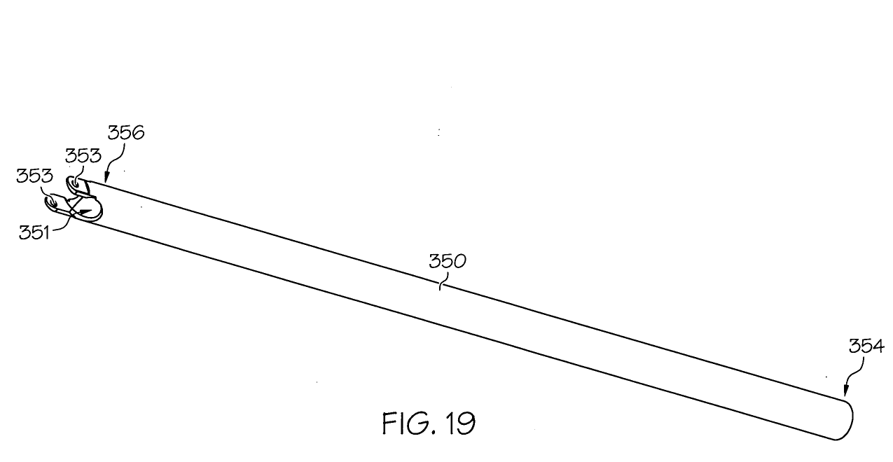

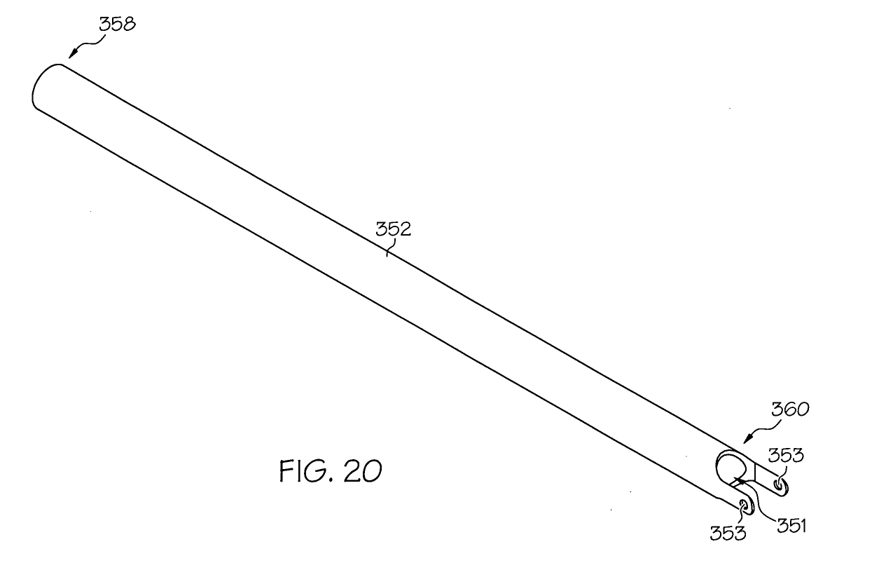

In various embodiments, a surgical instrument guide can include a first instrument receiving portion and a second instrument receiving portion. In at least one embodiment, referring to FIGS. 16-20, guide 310 can include first instrument receiving portion 350 and second receiving portion 352, wherein each of portions 350 and 352 can include an aperture, such as aperture 351, for example, configured to receive at least one surgical instrument therein. In various embodiments, these apertures can each include an instrument entry end and an instrument exit end. More particularly, referring to FIGS. 16 and 18, first portion 350 can include first instrument entry end 354 and first instrument exit end 356 and, similarly, second portion 352 can include second instrument entry end 358 and second instrument exit end 360. In at least one embodiment, instrument entry ends 354 and 358 can be configured to receive an end-effector of a surgical instrument from a position located outside of the patient's body and first and second instrument exit ends 356 and 360 can be configured to allow the end-effector to enter the patient's body at or near the tissue treatment region (FIG. 17). In various embodiments, first and second portions 350 and 352 can be comprised of a rigid, semi-rigid, and/or flexible material such as stainless steel, 6064 AL, polycarbonate and/or PEEK, for example, wherein the material can be selected in view of the flexibility needed for a particular surgical technique.

In various embodiments, the first and second portions of the surgical instrument guide can be movably connected to each other. In at least one embodiment, referring to FIGS. 18-20, first instrument exit end 356 of first portion 350 can be pivotably connected to second instrument exit end 360 of second portion 352 by connection member 362. In various embodiments, connection member 362 can include a pin, hinge, and/or any other suitable joint. In at least one embodiment, first portion 350 and second portion 352 can include projections extending therefrom and/or apertures, such as apertures 353, for example, therein configured to receive the projections. In various embodiments, these projections and apertures can be snap-fit together to retain first and second portions 350 and 352 together and permit relative movement therebetween. In any event, first portion 350 of guide 310 can be moved relative to second portion 352 between a first position and a second position. As outlined in greater detail below, first portion 350 can define a first axis and second portion 352 can define a second axis wherein the first axis and the second axis can be substantially collinear when portions 350 and 352 are in a first, undeployed, configuration. In at least one embodiment, the first and second axes may not be collinear when portions 350 and 352 are placed in a second, deployed, configuration. In various alternative embodiments, the first and second axes of portions 350 and 352 may not be substantially collinear when portions 350 and 352 are in their first configuration. In such embodiments, the first axis may be parallel, transverse, skew and/or perpendicular with respect to the second axis.

In various embodiments, surgical instrument guide 310 can be inserted into a patient's body at a first location and exit the patient's body at a second location. In at least one embodiment, guide 310 can be inserted through the anus of a patient and exit through a small incision in the abdominal wall “AW”. In other various embodiments, guide 310 can be inserted through the abdominal wall and can exit through the anus of the patient. In still other various embodiments, guide 310 can enter the patient through the anus and can exit through the oral cavity. In any event, a rigid, or at least substantially rigid, member can be inserted into guide 310 before guide 310 is inserted into the patient. In such embodiments, first and second portions 350 and 352 of guide 310 can remain in a substantially linear relative relationship when guide 310 is inserted into the patient's body. In various embodiments, surgical instrument guide 310 can be inserted into the patient such that instrument entry end 354 of first portion 350 and instrument entry end 358 of second portion 352 can be positioned outside of the patient's body.

In various embodiments, referring to FIGS. 16 and 17, surgical instrument guide 310 can further include holding members 374 which can be attached to external instrument entry ends 354 and 358 to assist a surgeon in holding guide 310 in place during the surgical procedure. In other various embodiments, holding members 374 can be positioned at any suitable location on first and second portions. In at least one embodiment, at least one of holding members 374 can be removably attached to guide 310 and can include a clamp configured to releasably grasp at least a portion of guide 310, for example. In various embodiments, in addition to or in place of holding members 374, guide 310 can be held in place by friction between guide 310 and the soft tissue, for example, surrounding a natural orifice and/or incision. In at least one embodiment, the outer side walls of guide 310 can include ridges and/or rough surfaces such that the coefficient of friction between the side walls of guide 310 and the portions of the patient's body can be increased to aid in holding guide 310 in position. In this fashion, surgical instrument guide 310 can remain substantially in position even when surgical instruments are slid through first and second portions 350 and 352 as described in greater detail below.

After surgical instrument guide 310 has been positioned within the patient's body as described above, the rigid member can be removed from guide 310 such that first portion 350 and second portion 352 can be moved relative to each other. In various embodiments, first portion 350 can be pivoted relative to second portion 352 such that instrument exit ends 356 and 360 can be rotated away from each other. In use, a surgeon can grasp first and second portions 350 and 352 and apply a force thereto to move them relative to each other. In other various embodiments, surgical instruments can be inserted into portions 350 and 352 such that a force can be applied to the surgical instruments to move portions 350 and 352 relative to each other. In either event, guide 310 can be configured such that a surgeon can pivot first portion 350 with respect to second portion 352 to create a suitable angle between the first and second axes thereof and properly align surgical instruments positioned within portions 350 and 352 within the surgical site. More particularly, referring to FIG. 17, end-effector 366 of first instrument 368 can be inserted through entry end 354 and exit end 356 of first portion 350 and, similarly, end-effector 370 of second instrument 372 can be inserted through entry end 358 and exit end 360 of second portion 352 such that surgical instruments 368 and 372 can be oriented at an angle relative to each other, for example. Once positioned in the surgical site, a surgeon can utilize instruments 368 and 372 to perform a desired surgical technique.

In various situations, depending on the orientation between first and second portions 350 and 352, one of surgical instruments 368 and 372 may have to be retracted in order to allow the other of surgical instruments 368 and 372 to pass thereby. These situations can typically arise when first and second portions 350 and 352 lie in substantially the same plane. In various embodiments, first and second portions 350 and 352 of guide 310 can be off-set from each other, i.e., lie in separate planes. In such embodiments, surgical instruments 368 and 372 can be moved relative to each other without contacting one another. In addition to or in lieu of the above, the apertures extending through first and second portions 350 and 352 can be configured such that at least one of surgical instruments 368 and 372 can be shifted, or displaced, such that instruments 368 and 372 can be moved relative to each other without having to retract one of the same. In at least one embodiment, at least one of the apertures can include a perimeter which is sufficiently larger than the perimeter and/or diameter of the surgical instrument positioned therein such that the surgical instrument can be shifted to one side of the aperture and then slid by the other surgical instrument. Stated another way, the aperture in first portion 350 can have a first inner perimeter and the aperture in second portion 352 can have a second inner perimeter, wherein the first inner perimeter can be larger than the second inner perimeter to allow the two surgical instruments to be simultaneously deployed into the tissue treatment region. In at least one embodiment, the aperture in first portion 350 can have a different shape than the aperture in second portion 352. For example, although not illustrated, the aperture in first portion 350 can have a rectangular cross-section which can be larger than a circular cross-section of the aperture in second portion 352. In various embodiments, the first and second portions can have any suitable cross-sectional shape, including a square, rectangle, circle, oval, and/or a triangle, for example. In at least one embodiment, the apertures of the first and second portions can each include sealing members positioned therein which can be configured to seal the tissue treatment region from an outside environment.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the invention described herein will be processed before surgery. First, a new or used instrument is obtained and if necessary cleaned. The instrument can then be sterilized. In one sterilization technique, the instrument is placed in a closed and sealed container, such as a plastic or TYVEK bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

While this invention has been described as having exemplary designs, the present invention may be further modified within the spirit and scope of the disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A surgical instrument guide configured to be at least partially inserted into a colonic cavity and anus of a patient during a surgical procedure, the surgical instrument guide comprising:
a body;
a first port in said body, wherein said first port is configured to receive at least a portion of a first surgical instrument, wherein said first port includes a first portion and a second portion, wherein said first portion defines a first perimeter, wherein said second portion defines a second perimeter, and wherein said first perimeter is larger than said second perimeter such that the first surgical instrument can be pivoted within said first port; and
a second port in said body, wherein said second port is configured to receive at least a portion of a second surgical instrument.

2. The surgical instrument guide of claim 1, wherein said first port includes a substantially conical chamfer comprising said first portion and said second portion.

3. The surgical instrument guide of claim 1, wherein said first port further includes a third portion, wherein said third portion defines a third perimeter, and wherein said third perimeter is larger than said second perimeter.

4. The surgical instrument guide of claim 3, wherein said first port includes a substantially conical chamfer comprising said second portion and said third portion.

5. The surgical instrument guide of claim 1, wherein at least one of said first and second ports further comprises a seal configured to sealingly engage one of the first and second surgical instruments within one of said first and second ports.

6. A surgical instrument guide configured to be at least partially inserted into a colonic cavity and anus of a patient during a surgical procedure, the surgical instrument guide comprising:
a first portion including a first port, wherein said first port is configured to receive at least a portion of a first surgical instrument;
a second portion including a second port, wherein said second port is configured to receive at least a portion of a second surgical instrument; and
a resilient member at least partially positioned intermediate said first portion and said second portion, wherein said first portion is movable relative to said second portion.

7. The surgical instrument guide of claim 6, wherein said first portion can be pivoted relative to said second portion.

8. The surgical instrument guide of claim 6, wherein said first portion further comprises at least one seal configured to sealingly engage the first surgical instrument within said first port.

9. A surgical instrument guide configured to be at least partially inserted into a colonic cavity and anus of a patient during a surgical procedure, the surgical instrument guide comprising:
a first portion configured to be positioned external to the colonic cavity and anus of the patient;
a second portion configured to be positioned within the colonic cavity of the patient, wherein said first portion and said second portion are configured to receive at least a portion of the anus intermediate said first portion and said second portion to retain said second portion in the colonic cavity; and
a port configured to receive at least a portion of a surgical instrument.

10. The surgical instrument guide of claim 9, further comprising a neck portion positioned intermediate said first portion and said second portion, wherein said neck portion is configured to receive at least a portion of said anus.

11. The surgical instrument guide of claim 10, wherein said first portion defines a first diameter, wherein said second portion defines a second diameter, and wherein said neck portion defines a diameter smaller than said first diameter and said second diameter.

12. The surgical instrument guide of claim 9, further comprising a groove configured to receive at least a portion of the anus.

13. The surgical instrument guide of claim 9, wherein said second portion is configured to be rotated within the colonic cavity.

14. The surgical instrument guide of claim 13, wherein said second portion is substantially spherical.

15. The surgical instrument guide of claim 9, further comprising a second port configured to receive at least a portion of a second surgical instrument.

16. The surgical instrument guide of claim 9, wherein said port further comprises at least one seal configured to sealingly engage the surgical instrument within said port.

17. A surgical instrument guide configured to be at least partially inserted into a colonic cavity and anus of a patient during a surgical procedure, the surgical instrument guide comprising:
a first portion, wherein said first portion is configured to receive at least a portion of a first surgical instrument; and
a second portion, wherein said second portion is configured to receive at least a portion of a second surgical instrument, and wherein said first portion is movably connected to said second portion.

18. The surgical instrument guide of claim 17, wherein said first portion is movable relative to said second portion between a first position and a second position, and wherein said first portion is one of collinear, parallel, transverse, skew, or perpendicular to said second portion when said first portion is in said second position.

19. The surgical instrument guide of claim 17, wherein said first portion includes an aperture configured to slidably receive the first surgical instrument, wherein said aperture includes a first end in which at least a portion of the first surgical instrument can enter the first portion, and wherein said aperture includes a second end in which at least a portion of the first surgical instrument can exit the first portion.

20. The surgical instrument guide of claim 17, wherein said first portion includes a first aperture configured to receive at least a portion of the first surgical instrument, wherein said first aperture includes a first perimeter, wherein said second portion includes a second aperture configured to receive at least a portion of the second surgical instrument, wherein said second aperture includes a second perimeter, and wherein said second perimeter is larger than said first perimeter to allow the second surgical instrument within said second aperture to be moved relative to the first surgical instrument within said first aperture.

21. The surgical instrument guide of claim 17, wherein said first portion further comprises a seal configured to sealingly engage the first surgical instrument within said first portion.

22. A surgical instrument guide configured to be at least partially inserted into a colonic cavity and anus of a patient during a surgical procedure, the surgical instrument guide comprising:
a first portion, wherein said first portion is configured to receive at least a portion of a first surgical instrument;
a second portion, wherein said second portion is configured to receive at least a portion of a second surgical instrument; and
pivot means for allowing pivotal movement between said first portion and said second portion.

23. The surgical instrument guide of claim 22, wherein said first portion is movable relative to said second portion between a first position and a second position, and wherein said first portion is one of collinear, parallel, transverse, skew, or perpendicular to said second portion when said first portion is in said first position.

24. The surgical instrument guide of claim 22, wherein said first portion includes an aperture configured to slidably receive the first surgical instrument, wherein said aperture includes a first end in which at least a portion of the first surgical instrument can enter the first portion, and wherein said aperture includes a second end in which at least a portion of the first surgical instrument can exit the first portion.

25. The surgical instrument guide of claim 22, wherein said first portion includes a first aperture configured to receive at least a portion of the first surgical instrument, wherein said first aperture includes a first perimeter, wherein said second portion includes a second aperture configured to receive at least a portion of the second surgical instrument, wherein said second aperture includes a second perimeter, and wherein said second perimeter is larger than said first perimeter to allow the second surgical instrument within said second aperture to be moved relative to the first surgical instrument within said first aperture.

26. The surgical instrument guide of claim 22, wherein said first portion further comprises a seal configured to sealingly engage the first surgical instrument within said first portion.

27. A surgical instrument guide configured to be at least partially inserted into a colonic cavity and anus of a patient during a surgical procedure, the surgical instrument guide comprising:
an elongate member movable between an undeployed configuration and a deployed configuration, wherein said elongate member comprises:
a first portion, wherein said first portion defines a first axis, and wherein said first portion is configured to receive a first surgical instrument; and
a second portion, wherein said second portion defines a second axis, wherein said second portion is configured to receive a second surgical instrument, wherein said first axis and said second axis are in a first relative relationship when said elongate member is in said undeployed configuration, and wherein said first axis and said second axis are in a second relative relationship when said elongate member is in said deployed configuration.

28. The surgical instrument guide of claim 27, wherein said first portion includes an aperture configured to slidably receive the first surgical instrument, wherein said aperture includes a first end in which at least a portion of the first surgical instrument can enter the first portion, wherein said aperture includes a second end in which at least a portion of the first surgical instrument can exit the first portion.

29. The surgical instrument guide of claim 27, wherein said first portion includes a first aperture configured to receive at least a portion of the first surgical instrument, wherein said first aperture includes a first perimeter, wherein said second portion includes a second aperture configured to receive at least a portion of the second surgical instrument, wherein said second aperture includes a second perimeter, and wherein said second perimeter is larger than said first perimeter to allow the second surgical instrument within said second aperture to be moved relative to the first surgical instrument within said first aperture.

30. The surgical instrument guide of claim 27, wherein said first portion further comprises a seal configured to sealingly engage the first surgical instrument within said first portion.

31. A surgical instrument guide configured to be at least partially inserted into a colonic cavity and anus of a patient during a surgical procedure, the surgical instrument guide comprising:
a body;
a first port in said body, wherein said first port defines a first axis, and wherein said first port is configured to receive at least a portion of a first surgical instrument; and
a second port in said body, wherein said second port defines a second axis, wherein said second port is configured to receive at least a portion of a second surgical instrument, and wherein said first axis is not parallel to said second axis.

32. The surgical instrument guide of claim 31, wherein said first port further comprises a seal configured to sealingly engage the first surgical instrument within said first port.
